(19) United States
(12) Patent Application Publication
(10) Pub. No.: US 2005/0272766 A1
(43) Pub. Date: Dec. 8, 2005

(54) 1-Glyoxylamide indolizines for treating lung and ovarian cancer

(75) Inventors: Keizo Koya, Chestnut Hill, MA (US); Lijun Sun, Harvard, MA (US); Hao Li, Brookline, MA (US); Mitsunori Ono, Lexington, MA (US)

Correspondence Address: HAMILTON, BROOK, SMITH & REYNOLDS, P.C., 530 VIRGINIA ROAD, P.O. BOX 9133, CONCORD, MA, 01742-9133, US

(73) Assignee: Synta Pharmaceuticals Corp., Lexington, MA (US)

(21) Appl. No.: 11/088,253

(22) Filed: Mar. 23, 2005

Related U.S. Application Data
(60) Provisional application No. 60/557,467, filed on Mar. 30, 2004.

(51) Int. Cl. A61K 31/4745

(52) U.S. Cl. 514/306

(57) ABSTRACT

A method of treating a subject having lung cancer or ovarian cancer, comprising administering to the subject an effective amount of a compound represented by Structural Formula (I) or a pharmaceutically acceptable salt, solvate, or polymorph thereof: 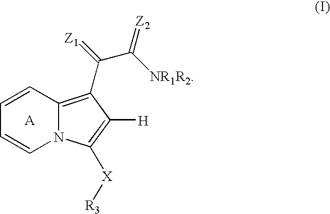 Ring A is substituted or unsubstituted and optionally fused to an aryl group.

Z₁ and Z₂ are independently ═O, ═S, ═N—OR₁₂ or ═NR₁₂

R₁ and R₂ are independently —H, an aliphatic group, a substituted aliphatic group, an unsubstituted non-aromatic heterocylic group, a substituted non-aromatic heterocylic group, an aryl group or a substituted aryl group, provided that R₁ and R₂ are not both —H. Alternatively, —NR₁R₂, taken together, is a substituted or unsubstituted non-aromatic nitrogen-containing heterocyclic group or a substituted or unsubstituted nitrogen-containing heteroaryl group.

R₃ is a substituted or unsubstituted aryl group or a substituted or unsubstituted aliphatic group.

X is a covalent bond, —C(R₄R₅)—, —N(R₄)—, —O—, —S—, —S(O)—, —S(O)₂—, —C(═O)—, —C(═O)—N(R₄)—, or —N(R₄)—C(═O)—.

R₄ and R₅ are independently —H or a substituted or unsubstituted aliphatic group.

R₁₂ is —H or a substituted or unsubstituted alkyl group.

### RELATED APPLICATIONS

This application claims the benefit of U.S. Provisional Application No. 60/557,467, filed on Mar. 30, 2004, the entire teachings of which are incorporated herein by reference.

### BACKGROUND OF THE INVENTION

Many methods are now available to be used in the treatment of cancer. Despite considerable advances, however, treatments for many cancers are inadequate for a number of reasons.

There are still cancers which simply do not respond or respond poorly to treatments are currently available. Patients with treatable cancers must often undergo chemotherapy with drugs that cause severe side effects. Few of these drugs can be used orally. Perhaps the most serious problem associated with cancer chemotherapy is the development of multi-drug resistance by many tumors. For example, many tumors which initially respond positively to an anti-cancer therapy by decreasing in size or even going into remission often develop resistance to the drug. Tumors that have developed resistance to more than one drug are said to be a “multi-drug resistant”. There is little that can be done to halt or retard further progression of the disease, once a patient's cancer has become multi-drug resistant.

There is therefore still a need for new drugs which overcome one or more of the aforementioned shortcomings of drugs currently used in the treatment of cancer. Desirable properties of new anti-cancer drugs therefore include efficacy against tumors that are currently untreatable or poorly treatable, efficacy against multi-drug resistant tumors, oral bioavailability and/or reduced side effects.

### SUMMARY OF THE INVENTION

It has now been found that 1-glyoxylamide indolizines are cytotoxic against cancer cells, including multi-drug resistant cancer cells, that cause lung cancer or ovarian cancer. For example, the percent inhibition of cancer cells by oral administration of Compound (1) was up to 61% for human ovarian cancer (BG1-TR1), and 80%, 67%, and 64% for human lung cancer (lines RERF-LC-AI, RERF/TXL, and Ma44, respectively; see Examples 1-4. 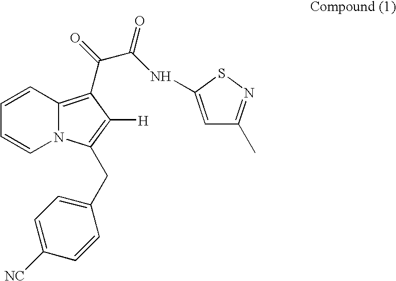
Minimal change in body weight was observed in mice treated with Compound 1, indicating that the compound caused minimal side-effects. These results compare favorably or exceed the results obtained using Taxol as a positive control. Based on these results, methods of treating subjects with lung cancer or ovarian cancer by administering 1-glyoxylamide indolizines are disclosed herein.

A method of treating a subject having lung cancer or ovarian cancer comprises administering to the subject an effective amount of a compound represented by Structural Formula (I) or a pharmaceutically acceptable salt, solvate, or polymorph thereof:

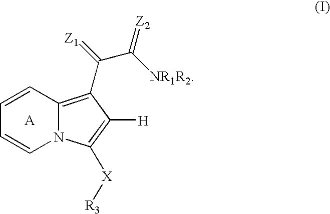

Ring A is substituted or unsubstituted and is optionally fused to an aryl group (preferably substituted or unsubstituted phenyl).

Z₁ and Z₂ are independently ═O, ═S, ═N—OR₁₂ or ═NR₁₂.

R₁ and R₂ are independently —H, an aliphatic group, a substituted aliphatic group, an unsubstituted non-aromatic heterocylic group, a substituted non-aromatic heterocylic group, an unsubstituted aryl group or a substituted aryl group, provided that R₁ and R₂ are not both —H. Alternatively, —NR₁R₂, taken together, is a substituted or unsubstituted non-aromatic nitrogen-containing heterocyclic group or a substituted or unsubstituted nitrogen-containing heteroaryl group.

R₃ is a substituted or unsubstituted aryl group or a substituted or unsubstituted aliphatic group.

X is a covalent bond, —C(₄R₅)—, —N(R₄)—, —O—, —S—, —S(O)—, —S(O)₂—, —C(═O)—, —C(═O)—N(R₄)—, or —N(R₄)—C(═O)—.

R₄ and R₅ are independently —H or a substituted or unsubstituted aliphatic group.

R₁₂ is —H or a substituted or unsubstituted alkyl group.

Another embodiment is a method of treating a subject with cancer. The method comprises administering to the subject an effective amount of a compound represented by Structural Formula (I).

The disclosed methods employing 1-glyoxylamide indolizines have many advantages when used to treat lung and ovarian cancer. Most significantly, they are cytotoxic to many multi-drug resistant cell lines and therefore can be used when other traditional cancer chemotherapies have failed. In addition, they exhibit minimal side effects and are active when administered orally.

### DETAILED DESCRIPTION OF THE INVENTION

In a preferred embodiment, the compound employed in the present invention is represented by Structural Formula (I) wherein Ring A is a substituted or unsubstituted aryl group; Z₁ and Z₂ are both ═O; R₁ is —H; R₂ is a substituted or unsubstituted alkyl or aryl group; R₃ is a substituted or unsubstituted aryl group; X is —C(R₄R₅)—, —N(R₄)— or —O— (preferably, X is —C(R₄R₅)—); and R₄, R₅, Z₁ and Z₂ are as described above. More preferably, R₃ is a substituted or unsubstituted phenyl or pyridyl group; and R₄ and R₅ are both —H.

As noted above, values for R₁-R₃ include substituted and unsubstituted aryl group. For R₂, preferred aryl groups are represented by Structural Formulas (II)-(XV): 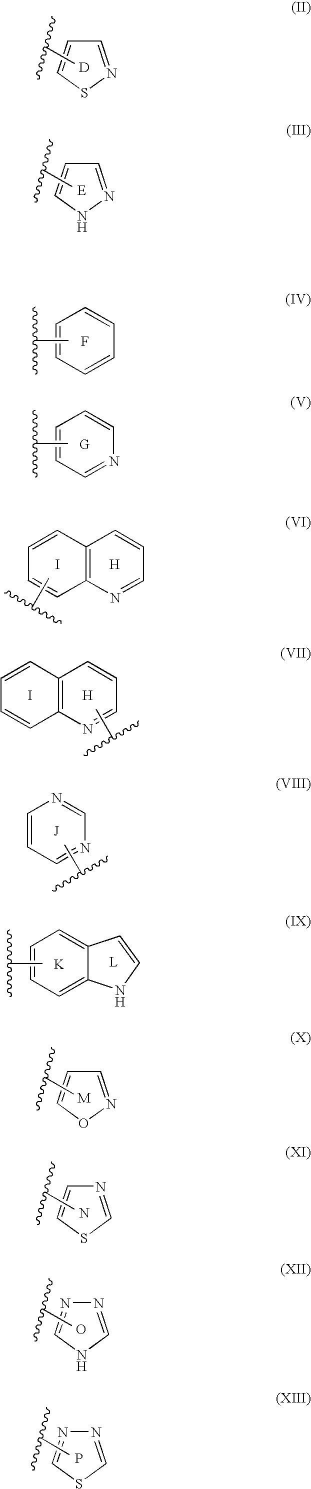 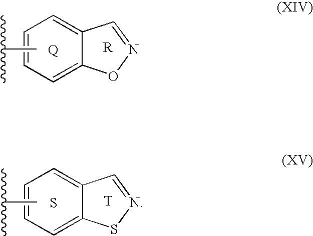
Rings D-T are substituted or unsubstituted. More preferred aryl groups for R₂ are represented by Structural Formulas (XVI)-(XXI):

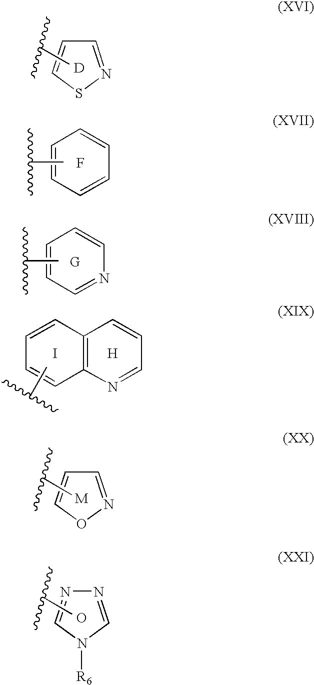

R₆ is —H or a substituted or unsubstituted alkyl group and Rings D, F, G, I, H, M and O are as described above.

Even more preferred aryl groups for R₂ are represented by Structural Formulas (XXII)-(XXVII):

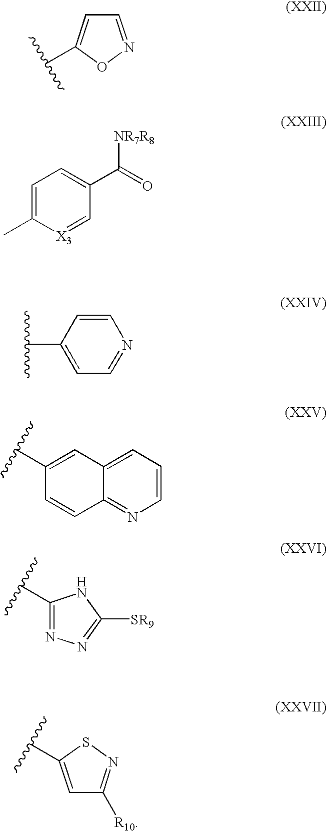

X₃ is —CH— or —N—.

R₇ and R₈ are independently —H or an alkyl group. Alternatively, —NR₇R₈, taken together, is a nitrogen-containing non-aromatic heterocyclic group.

R₉ is an alkyl group.

R₁₀ is —H or an alkyl group.

In another preferred embodiment, the compound of the present invention is represented by Structural Formula (XXVIII):

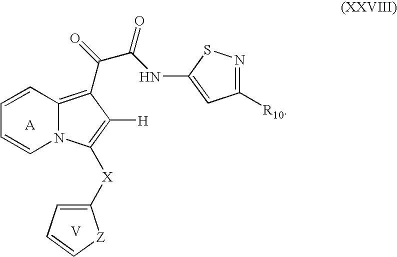

In Structural Formula (XXVIII), Rings A and V are independently substituted or unsubstituted; X is —CH₂—, —CH(CH₃)—, —O—, —NH— or —NCH₃—; Z is —O—, —S—, —NR—, —C═C—, —CH═N—, —N═CH—, —N═N—; R is —H or C1-C4 alkyl; and R₁₀ is —H, an unsubstituted aliphatic group or a substituted aliphatic group. Even more preferably, Ring A is unsubstituted, Ring V is substituted with one or more groups represented by R₁₁, wherein each R₁₁ is independently —CH₃, —CH₂CH₃, —OCH₃, —F, —Cl or —CN.

In a more preferred embodiment, the compound of the present invention is represented by Structural Formula (XXIX):

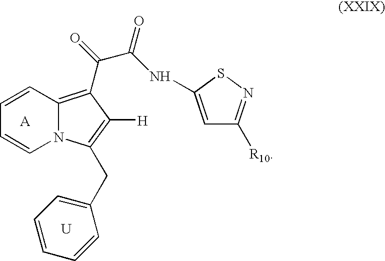

In Structural Formula (XXIX), Rings A and U are independently substituted or unsubstituted and R₁₀ is —H, an unsubstituted aliphatic group or a substituted aliphatic group. Even more preferably, Ring A is unsubstituted, Ring U is substituted with one or more groups represented by R₁₁, wherein each R₁₁ is independently —CH₃, —CH₂CH₃, —OCH₃, —F, —Cl or —CN. Preferably, R₁₁ is in the para position relative to the carbon bonded to the methylene group.

In other embodiments, preferred compounds include one or more of compounds

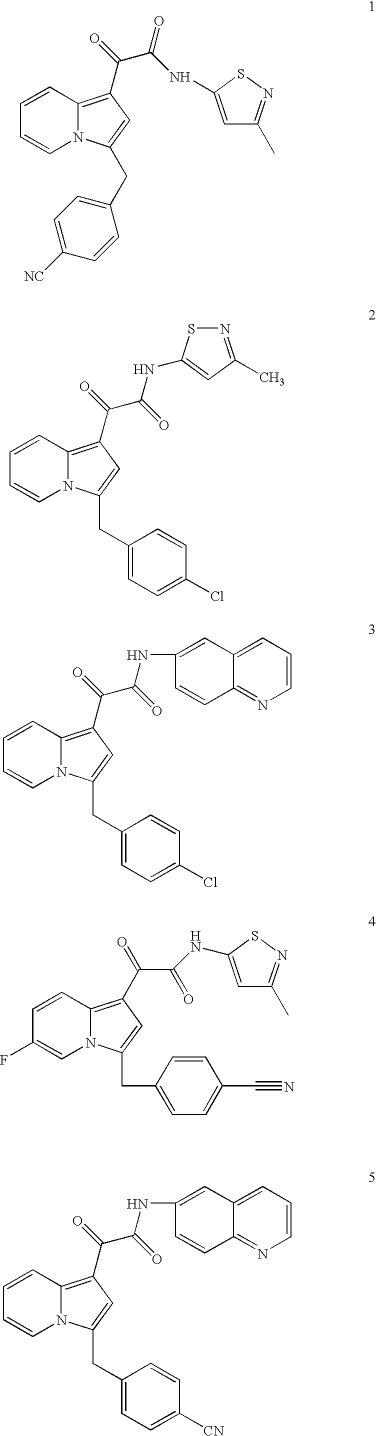

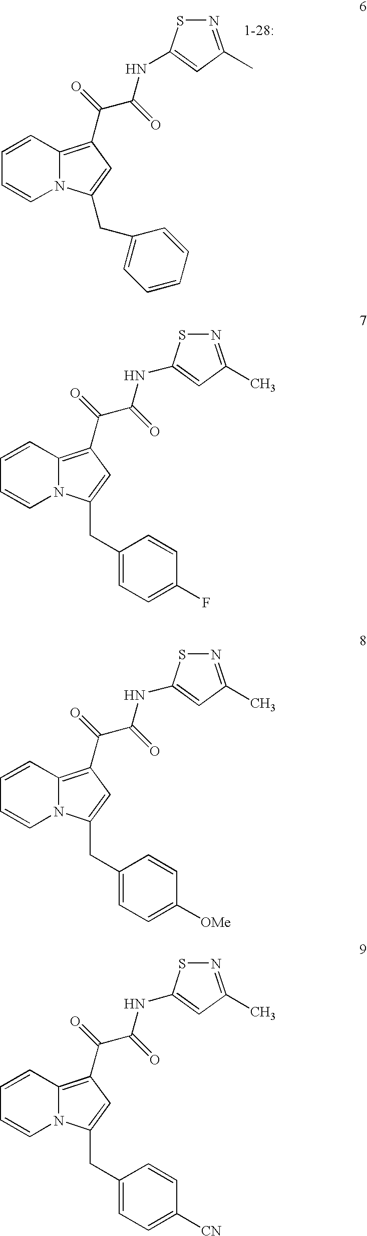

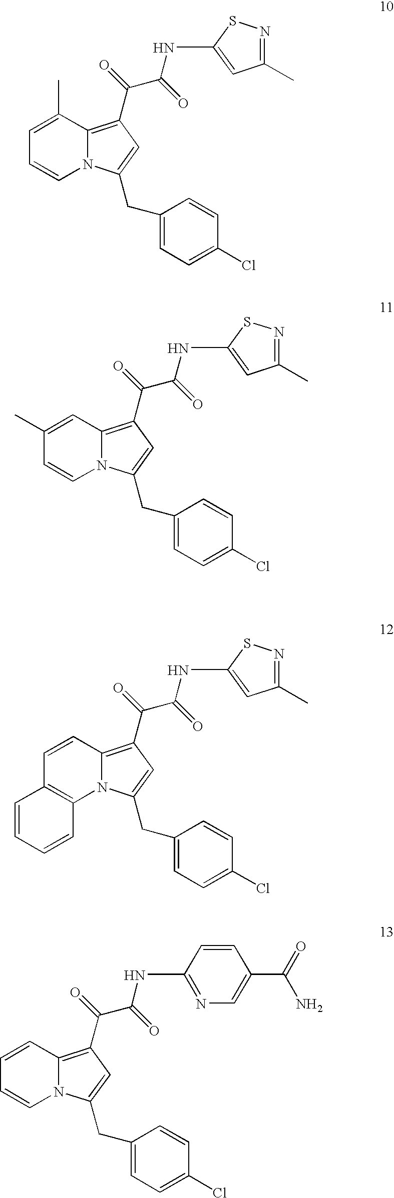

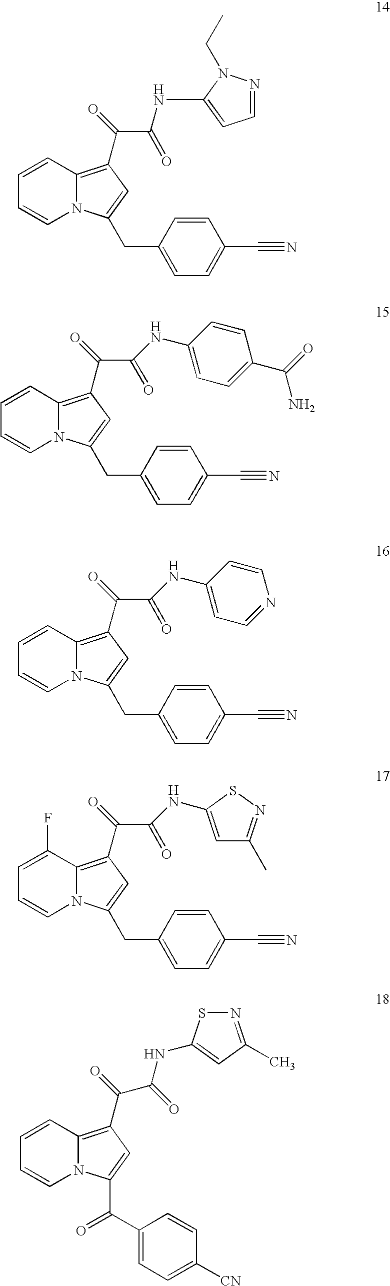

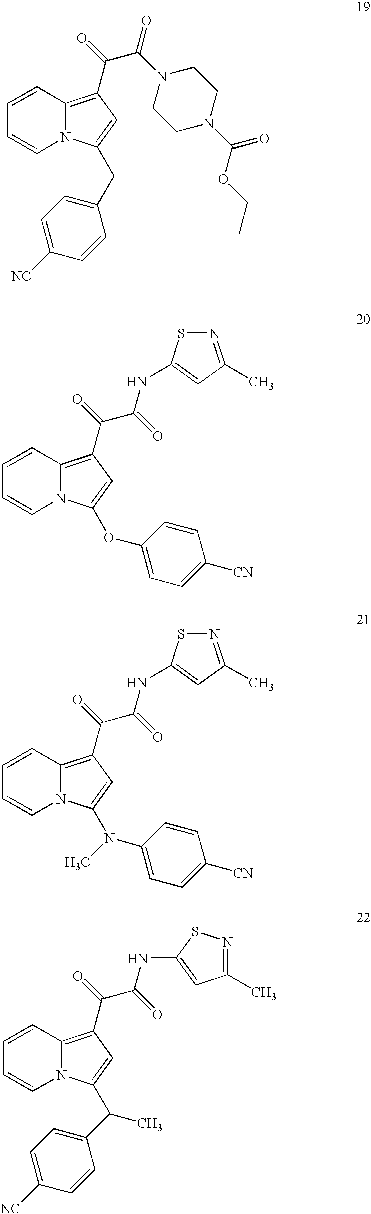

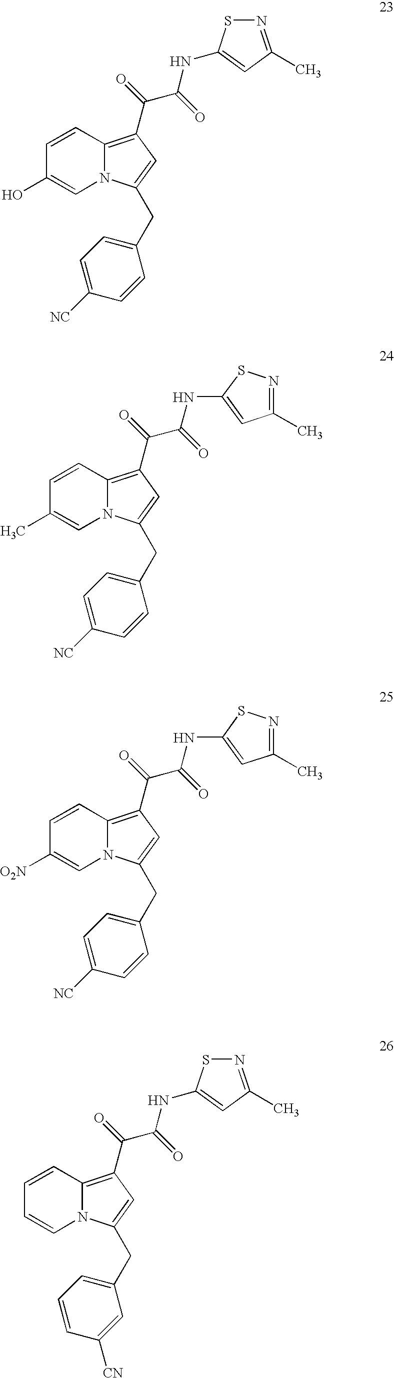

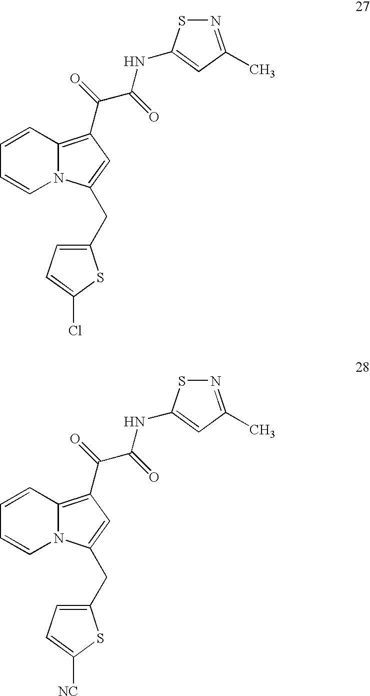

In other embodiments, preferred compounds include one or more of:
2-(3-(4-cyanobenzyl)indolizin-1-yl)-N-(3-methylisothiazol-5-yl)-2-oxoacetamide;
2-(3-(4-chlorobenzyl)indolizin-1-yl)-N-(3-methylisothiazol-5-yl)-2-oxoacetamide;
2-(3-(4-chlorobenzyl)indolizin-1-yl)-2-oxo-N-(quinolin-6-yl)acetamide;
2-(3-(4-cyanobenzyl)-6-fluoroindolizin-1-yl)-N-(3-methylisothiazol-5-yl)-2-oxoacetamide;
2-(3-(4-cyanobenzyl)indolizin-1-yl)-2-oxo-N-(quinolin-6-yl)acetamide;
2-(3-benzylindolizin-1-yl)-N-(3-methylisothiazol-5-yl)-2-oxoacetamide;
2-(3-(4-fluorobenzyl)indolizin-1-yl)-N-(3-methylisothiazol-5-yl)-2-oxoacetamide;
2-(3-(4-methoxybenzyl)indolizin-1-yl)-N-(3-methylisothiazol-5-yl)-2-oxoacetamide;
2-(3-(4-cyanobenzyl)indolizin-1-yl)-N-(3-methylisothiazol-5-yl)-2-oxoacetamide;
2-(3-(4-chlorobenzyl)-8-methylindolizin-1-yl)-N-(3-methylisothiazol-5-yl)-2-oxoacetamide;
2-(3-(4-chlorobenzyl)-7-methylindolizin-1-yl)-N-(3-methylisothiazol-5-yl)-2-oxoacetamide;
2-(1-(4-chlorobenzyl)pyrrolo[1,2-a]quinolin-3-yl)-N-(3-methylisothiazol-5-yl)-2-oxoacetamide;
2-(3-(4-chlorobenzyl)indolizin-1-yl)-N-(2-carboxamidopyridin-5-yl)-2-oxoacetamide;
2-(3-(4-cyanobenzyl)indolizin-1-yl)-N-(1-ethyl-1H-pyrazol-5-yl)-2-oxoacetamide;
2-(3-(4-cyanobenzyl)indolizin-1-yl)-N-(4-phenylamide)-2-oxoacetamide;
2-(3-(4-cyanobenzyl)indolizin-1-yl)-2-oxo-N-(pyridin-4-yl)acetamide;
2-(3-(4-cyanobenzyl)-8-fluoroindolizin-1-yl)-N-(3-methylisothiazol-5-yl)-2-oxoacetamide;
2-(3-(((4-cyano)-phenyl)carbonyl)indolizin-1-yl)-N-(3-methylisothiazol-5-yl)-2-oxoacetamide;
2-(3-((4-cyano)-phenylmethyl)indolizin-1-yl)-1-(4-ethoxycarbonylpiperazin-1-yl)ethane-1,2-dione;
2-(3-(4-cyanophenoxy)indolizin-1-yl)-N-(3-methylisothiazol-5-yl)-2-oxoacetamide;
2-(3-(((4-cyano)-phenyl)methylamino)indolizin-1-yl)-N-(3-methylisothiazol-5-yl)-2-oxoacetamide;
2-(3-(1-(4-cyanophenyl)ethyl)indolizin-1-yl)-N-(3-methylisothiazol-5-yl)-2-oxoacetamide;
2-(3-(4-cyanobenzyl)-6-hydroxyindolizin-1-yl)-N-(3-methylisothiazol-5-yl)-2-oxoacetamide;
2-(3-(4-cyanobenzyl)-6-methylindolizin-1-yl)-N-(3-methylisothiazol-5-yl)-2-oxoacetamide;
2-(3-(4-cyanobenzyl)-6-nitroindolizin-1-yl)-N-(3-methylisothiazol-5-yl)-2-oxoacetamide;
2-(3-(3-cyanobenzyl)indolizin-1-yl)-N-(3-methylisothiazol-5-yl)-2-oxoacetamide;
2-(3-((5-chlorothiophen-2-yl)methyl)indolizin-1-yl)-N-(3-methylisothiazol-5-yl)-2-oxoacetamide; or
2-(3-((5-cyanothiophen-2-yl)methyl)indolizin-1-yl)-N-(3-methylisothiazol-5-yl)-2-oxoacetamide.

Synthetic methods for the preparation of the 1-glyoxylindolizines represented by Structural Formula (I), e.g., compounds (1)-(28) are described in detail in Keizo, et al, U.S. Patent Application 20030153759, published Aug. 14, 2003, the entire teachings of which are incorporated herein by reference.

The term “aryl group” refers to carbocyclic aromatic groups such as phenyl, naphthyl, and anthracyl, and heteroaryl groups such as imidazolyl, isoimidazolyl, thienyl, furanyl, pyridyl, pyrimidyl, pyranyl, pyrazolyl, pyrrolyl, pyrazinyl, thiazoyl, isothiazolyl, oxazolyl, isooxazolyl, 1,2,3-trizaolyl, 1,2,4-triazolyl, and tetrazolyl.

Aryl groups also include fused polycyclic aromatic ring systems in which a carbocyclic aromatic ring or heteroaryl ring is fused to one or more other heteroaryl rings. Examples include benzothienyl, benzofuranyl, indolyl, quinolinyl, benzothiazolyl, benzoisothiazolyl, benzooxazolyl, benzoisooxazolyl, benzimidazolyl, quinolinyl, isoquinolinyl and isoindolyl.

An aliphatic group is a straight chained, branched or cyclic non-aromatic hydrocarbon which is completely saturated or which contains one or more units of unsaturation. Typically, a straight chained or branched aliphatic group has from 1 to about 10 carbon atoms, preferably from 1 to about 4, and a cyclic aliphatic group has from 3 to about 10 carbon atoms, preferably from 3 to about 8. An aliphatic group is preferably a straight chained or branched alkyl group, e.g, methyl, ethyl, n-propyl, iso-propyl, n-butyl, sec-butyl, tert-butyl, pentyl, hexyl, pentyl or octyl, or a cycloalkyl group with 3 to about 8 carbon atoms. A C1-C4 straight chained or branched alkyl group or a C3-C8 cyclic alkyl group is also referred to as a “lower alkyl” group.

An “alkylene group” is represented by —(CH₂)ₙ—. n is an integer from 1-10, preferably 1-4.

Non-aromatic heterocyclic rings are non-aromatic carbocyclic rings which include one or more heteroatoms such as nitrogen, oxygen or sulfur in the ring. The ring can be five, six, seven or eight-membered. Examples include oxazolinyl, thiazolinyl, oxazolidinyl, thiazolidinyl, tetrahydrofuranyl, tetrahyrothiophenyl, morpholino, thiomorpholino, pyrrolidinyl, piperazinyl, piperidinyl, and thiazolidinyl.

Suitable substituents for an aliphatic group, non-aromatic heterocyclic group, benzylic or an aryl group ring carbon (carbocyclic and heteroaryl) are those which do not substantially interfere with the anti-cancer activity of the compounds represented by Structural Formula I. Examples of suitable substituents include —OH, halogen (—Br, —Cl, —I and —F), —OR^{a}, —O—COR^{a}, —COR^{a}, —CN, —NO₂, —COOH, —SO₃H, —NH₂, —NHR^{a}, —N(R^{a}R^{b}), —COOR^{a}, —CHO, —CONH₂, —CONHR^{a}, —CON(R^{a}R^{b}), —NHCOR^{a}, —NRCOR^{a}, —NHCONH₂, —NHCONR^{a}H, —NHCON(R^{b}), —NRCCONH₂, —NR^{c}CONR^{a}H, —NR^{c}CON(R^{a}R^{b}), —C(═NH)—NH₂, —C(═NH)—NHR^{a}, —C(═NH)—N(R^{a}R^{b}), —C(═NR^{c})—NH₂, —C(═NR^{c})—NHR^{a}, —C(═NR^{c})—N(R^{a}R^{b}), —NH—C(═NH)—NH₂, —NH—C(═NH)—NHR^{a}, —NH—C(═NH)—N(R^{a}R^{b}), —NH—C(═NR^{c})—NH₂, —NH—C(═NR^{c})—NHR^{a}, —NH—C(═NR^{c})—N(R^{a}R^{b}), —NR^{d}H—C(═NH)—NH₂, —NR^{d}—C(═NH)—NHR^{a}, —NR^{d}—C(═NH)—N(R^{a}R^{b}), —NR^{d}—C(═NR^{c})—NH₂, —NR^{d}—C(═NR^{c})—NHR^{a}, —NR^{d}—C(═NR^{c})—N(R^{a}R^{b}), —NHNH₂, —NHNHR^{a}, —NHR^{a}R^{b}, —SO₂NH₂, —SO₂NHR^{a}, —SO₂NR^{a}R^{b}, —CH═CHR^{a}, —CH═CR^{a}R^{b}, —CR^{c}═CR^{a}R^{b}—CR^{c}═CHR^{a}, —CR^{c}═CR^{a}R^{b}, —CCR^{a}, —SH, —SOₖR^{a} (k is 0, 1 or 2) and —NH—C(═NH)—NH₂. R^{a}-R^{d} are each independently an aliphatic, substituted aliphatic, benzyl, substituted benzyl, aryl or substituted aryl group, preferably an alkyl, benzylic or aryl group. In addition, —N(R^{a}R^{b}), taken together, can also form a substituted or unsubstituted non-aromatic heterocyclic group. A non-aromatic heterocyclic group, benzylic group or aryl group can also have an aliphatic or substituted aliphatic group as a substituent. A substituted aliphatic group can also have a non-aromatic heterocyclic ring, a substituted a non-aromatic heterocyclic ring, benzyl, substituted benzyl, aryl or substituted aryl group as a substituent. A substituted aliphatic, non-aromatic heterocyclic group, substituted aryl, or substituted benzyl group can have more than one substituent.

Suitable substituents for heteroaryl ring nitrogen atoms having three covalent bonds to other heteroaryl ring atoms include —OH and -alkoxy (preferably C1-C4). Substituted heteroaryl ring nitrogen atoms that have three covalent bonds to other heteroaryl ring atoms are positively charged, which is balanced by counteranions such as chloride, bromide, formate, acetate and the like. Examples of other suitable counteranions are provided in the section below directed to suitable pharmacologically acceptable salts.

Suitable substituents for heteroaryl ring nitrogen atoms having two covalent bonds to other heteroaryl ring atoms include alkyl, substituted alkyl (including haloalkyl), phenyl, substituted phenyl, —S(O)₂-(alkyl), —S(O)₂—NH(alkyl) and —S(O)₂—NH(alkyl)₂.

Preferred substituents for Ring A include —F, —Cl, —Br, —C1-C4 alkyl, C1-C4 alkoxy, —C1-C4 haloalkyl, C1-C4 haloalkoxy, —CN or —NH₂. Ring A can have zero, one or more substituents.

Preferred substituents for Rings D-T include C1-C4 alkyl, C1-C4 hydroxyalkyl, N-morpholino, pyrimidyl, C1-C4 alkyl substituted with pyrimidyl, —N(C1-C4 alkyl)₂, —C(O)NH₂, —C(O)NH(C1-C4 alkyl), C(O)N(C1-C4 alkyl)₂, —NHC(O)(C1-C4 alkyl), —NO₂, C1-C4 alkoxy, —C(O)O—CH₂CH₂—N(C1-C4 alkyl)₂, 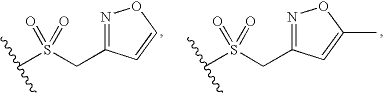
—NH-phenyl), —NH₂, —CH₂NH—C(O)—O—(C1-C4 alkyl), —CH₂NH₂, —Cl, —F, —C(O)—O—(C1-C4 alkyl), —C(O)—N—(C1-C4 alkyl), C3-C7 cycloalkyl, phenyl, —C(O)—N-morpholino, —S—(C1-C4 alkyl), —CN, furyl, —S(O)₂—(C1-C4 alkyl), —S(O)₂—NH₂, —S(O)₂—NH(C1-C4 alkyl) or —S(O)₂—N(C1-C4 alkyl)₂.

Preferred substituents for Ring U, Ring V and the phenyl and pyridyl ring represented by R₃ include —Br, —Cl, —F, —Re, —OR^{e}, —CN, —COOR^{e}, —N(R^{e})₂, —CON(R^{e})₂, —NR^{e}COR^{f}, —NHCONH₂ and —SO₂N(R^{e})₂. Each R^{e} and R^{f} are independently selected from —H, alkyl or substituted alkyl. More preferred substituents for Ring U, Ring V and the phenyl group represented by R₃ include —Cl, —F, —R^{e}, —OR^{e}, —CN, —NH₂, —CONH₂ or —NHCOR^{f}. Even more preferred substituents for Ring U, Ring V and the phenyl group represented by R₃ include —CH₃, —CH₂CH₃, —F, —Cl, —CN or —OCH₃. Ring U, Ring V and the phenyl and pyridyl ring represented by R₃ can have zero, one or more substituents, but are preferably unsubstituted or monosubstituted substituted. When Ring U, Ring V and R₃ are a six-membered aromatic ring and monosubstituted, the substituent is preferably at the position para to the carbon atom bonded to the methylene group.

Also included in the present invention are pharmaceutically acceptable salts and solvates of the compounds described herein. The compounds represented by Structural Formula I which possess a sufficiently acidic, a sufficiently basic, or both functional groups, and accordingly can react with any of a number of organic or inorganic bases, and inorganic and organic acids, to form a salt. Acids commonly employed to form acid addition salts from compounds with basic groups are inorganic acids such as hydrochloric acid, hydrobromic acid, hydroiodic acid, sulfuric acid, phosphoric acid, and the like, and organic acids such as p-toluenesulfonic acid, methanesulfonic acid, oxalic acid, p-bromophenyl-sulfonic acid, carbonic acid, succinic acid, citric acid, benzoic acid, acetic acid, and the like. Examples of such salts include the sulfate, pyrosulfate, bisulfate, sulfite, bisulfite, phosphate, monohydrogenphosphate, dihydrogenphosphate, metaphosphate, pyrophosphate, chloride, bromide, iodide, acetate, propionate, decanoate, caprylate, acrylate, formate, isobutyrate, caproate, heptanoate, propiolate, oxalate, malonate, succinate, suberate, sebacate, fumarate, maleate, butyne-1,4-dioate, hexyne-1,6-dioate, benzoate, chlorobenzoate, methylbenzoate, dinitrobenzoate, hydroxybenzoate, methoxybenzoate, phthalate, sulfonate, xylenesulfonate, phenylacetate, phenylpropionate, phenylbutyrate, citrate, lactate, gamma-hydroxybutyrate, glycolate, tartrate, methanesulfonate, propanesulfonate, naphthalene-1-sulfonate, naphthalene-2-sulfonate, mandelate, and the like.

Base addition salts include those derived from inorganic bases, such as ammonium or alkali or alkaline earth metal hydroxides, carbonates, bicarbonates, and the like. Such bases useful in preparing the salts of this invention thus include sodium hydroxide, potassium hydroxide, ammonium hydroxide, potassium carbonate, and the like.

The compounds represented by Structural Formula I can be used to treat subjects with cancer, including multi-drug resistant cancers. A cancer is resistant to a drug when it resumes a normal rate of tumor growth while undergoing treatment with the drug after the tumor had initially responded to the drug. A tumor “responds to a drug” when it exhibits a decrease in tumor mass or a decrease in the rate of tumor growth. The term “multi-drug resistant cancer” refers to cancer that is resistant to two or more drugs, typically five or more.

Cancers treatable by the present invention include lung cancer (e.g., primary and secondary, generally primary lung cancer) and ovarian cancer.

Ovarian cancers treatable by the present invention typically include epithelial ovarian carcinoma (EOC) (for example, serous, endometriod, mucinous, clear cell, undifferentiated, borderline tumors, and the like), germ cell carcinoma (teratomas, dysgerminomas, endodermal sinus tumors, and the like), and stromal carcinoma (granulosa cell tumors, Sertoli-Leydig cell tumors, and the like). More preferably, treatable ovarian cancers are BG type carcinomas, e.g., BG1/BG2 carcinomas, most preferably BG1-TR1 carcinoma.

In some embodiments, primary lung cancer is treatable, generally including, for example, epithelial tumors: benign, e.g., papillomas and adenomas; dysplasia/carcinoma in situ; malignant, e.g., squamous cell carcinoma (including spindle cell variant), small cell carcinoma (including, for example, oat cell carcinoma, intermediate cell type, and combined oat cell carcinoma), adenocarcinoma (for example, acinar, papillary, bronchioalveolar, solid carcinoma with and without mucin formation, bronchial surface epithelial cell type, goblet cell type, Clara cell type, type II alveolar epithelial cell type, and bronchial gland cell type), large cell carcinoma (including, for example, giant cell carcinoma, large cell anaplastic carcinoma, large cell neuroendocrine carcinoma, and clear cell carcinoma), adenosquamous carcinoma, typical carcinoid, atypical carcinoid, bronchial gland carcinoma, and the like. Also included are combinations of the preceding, e.g., small cell carcinoma with large cell component, and combined small cell carcinoma with either adenocarcinoma or squamous cell carcinoma, e.g., combinations of squamous and glandular elements, called adenosquamous cell carcinoma. Also included are soft tissue tumors and mesothelial tumors, both benign and malignant, generally malignant. Further included are those cancers currently unclassified that can be recognized by one skilled in the art as lung cancers. In some embodiments, lung cancers treatable by the instant invention include non-small cell lung cancers (e.g., large cell carcinomas) and squamous cell carcinomas. More typically, squamous cell carcinomas include RERF cell lines, e.g., RERF-LC-AI and RERF/TXL, and the like, and non-small cell lung cancers include Ma44 cell lines, and the like. In various individual embodiments, lung cancers treatable by the present invention include a single cell line selected from RERF-LC-AI, RERF/TXL, and Ma44.

Thus, in some embodiments, the subject, typically a human subject, has (e.g., is treated for) human primary lung cancer or human ovarian cancer. In other embodiments, the subject has ovarian cancer, typically epithelial ovarian carcinoma, ovarian germ cell carcinoma, or ovarian stromal carcinoma, more typically ovarian BG type carcinoma, or preferably ovarian BG1-TR1 carcinoma. In individual preferred embodiments, the subject has one of epithelial ovarian carcinoma, ovarian germ cell carcinoma, or ovarian stromal carcinoma. In other embodiments, the subject has squamous cell carcinoma, small cell carcinoma, adenocarcinoma, adenosquamous carcinoma, or large cell carcinoma, more typically squamous cell carcinoma or large cell carcinoma. In individual preferred embodiments, the subject has one of squamous cell carcinoma, small cell carcinoma, adenocarcinoma, adenosquamous carcinoma, or large cell carcinoma. In other individual preferred embodiments, the subject has one of RERF-LC-AI type carcinoma, RERF/TXL type carcinoma, or Ma44 type carcinoma.

An “effective amount” is the quantity of compound in which a beneficial clinical outcome is achieved when the compound is administered to a subject with a multi-drug resistant cancer. A “beneficial clinical outcome” includes a reduction in tumor mass, a reduction in the rate of tumor growth, a reduction in metastasis, a reduction in the severity of the symptoms associated with the cancer and/or an increase in the longevity of the subject compared with the absence of the treatment. The precise amount of compound administered to a subject will depend on the type and severity of the disease or condition and on the characteristics of the subject, such as general health, age, sex, body weight and tolerance to drugs. It will also depend on the degree, severity and type of cancer. The skilled artisan will be able to determine appropriate dosages depending on these and other factors. Effective amounts of the compounds represented by Structural Formula I typically range between about 1 mg/mm² per day and about 10 grams/mm² per day, and preferably between 10 mg/mm² per day and about 5 grams/mm².

The compounds represented by Structural Formula I are administered by any suitable route, including, for example, orally in capsules, suspensions or tablets or by parenteral administration. Parenteral administration can include, for example, systemic administration, such as by intramuscular, intravenous, subcutaneous, or intraperitoneal injection. The compounds can also be administered orally (e.g., dietary), topically, by inhalation (e.g., intrabronchial, intranasal, oral inhalation or intranasal drops), or rectally, depending on the type of cancer to be treated. Oral or parenteral administration are preferred modes of administration.

The compounds employed in the invention may contain one or more chiral centers or double bonds and, therefore, exist as stereoisomers, such as double-bond isomers (i.e., geometric isomers), enantiomers, or diastereomers. According to this invention, the chemical structures depicted herein, including the compounds of this invention, encompass all of the corresponding compounds' enantiomers and stereoisomers, that is, both the stereomerically pure form (e.g., geometrically pure, enantiomerically pure, or diastereomerically pure) and enantiomeric and stereoisomeric mixtures. In some cases, one enantiomer or stereoisomer will possess superior activity or an improved toxicity or kinetic profile compared to others. In those cases, such enantiomers and stereoisomers of compounds of this invention are preferred.

As used herein, the term “polymorph” means to solid crystalline forms of a compound of the present invention or complex thereof. Different polymorphs of the same compound can exhibit different physical, chemical or spectroscopic properties. Different physical properties include, but are not limited to stability (e.g., to heat or light), compressibility and density (important in formulation and product manufacturing), and dissolution rates (which can affect bioavailability). Differences in stability can result from changes in chemical reactivity (e.g., differential oxidation, such that a dosage form discolors more rapidly when comprised of one polymorph than when comprised of another polymorph) or mechanical characteristics (e.g., tablets crumble on storage as a kinetically favored polymorph converts to thermodynamically more stable polymorph) or both (e.g., tablets of one polymorph are more susceptible to breakdown at high humidity). Different physical properties of polymorphs can affect their processing. For example, one polymorph might be more likely to form solvates or might be more difficult to filter or wash free of impurities than another due to, for example, the shape or size distribution of particles of it.

As used herein, the term “solvate” means a compound of the present invention or a salt thereof, that further includes a stoichiometric or non-stoichiometric amount of solvent, e.g., water or organic solvent, bound by non-covalent intermolecular forces.

The compounds represented by Structural Formula I can be administered to the subject in conjunction with an acceptable pharmaceutical carrier as part of a pharmaceutical composition for treatment of cancer. Formulation of the compound to be administered will vary according to the route of administration selected (e.g., solution, emulsion, capsule). Suitable pharmaceutical carriers may contain inert ingredients which do not interact with the compound. Standard pharmaceutical formulation techniques can be employed, such as those described in Remington's Pharmaceutical Sciences, Mack Publishing Company, Easton, Pa. Suitable pharmaceutical carriers for parenteral administration include, for example, sterile water, physiological saline, bacteriostatic saline (saline containing about 0.9% mg/ml benzyl alcohol), phosphate-buffered saline, Hank's solution, Ringer's-lactate and the like. Methods for encapsulating compositions (such as in a coating of hard gelatin or cyclodextrasn) are known in the art (Baker, et al., “Controlled Release of Biological Active Agents”, John Wiley and Sons, 1986).

Typically, the method employs one compound represented by Structural Formula I as a monotherapy. In other embodiments, the compounds represented by Structural Formula I can be co-administered with other anti-cancer agents. In some embodiments, the compounds are preferably co-administered before the cancer develops multi-drug resistance or as the cancer is developing multi-drug resistance but before the cancer becomes completely resistant to the anticancer drugs being used. The method can also be carried in combination with other cancer treatments such as surgery, radiation, and the like.

Other anti-cancer agents include, for example, Adriamycin, Dactinomycin, Bleomycin, Vinblastine, Cisplatin, acivicin; aclarubicin; acodazole hydrochloride; acronine; adozelesin; aldesleukin; altretamine; ambomycin; ametantrone acetate; aminoglutethimide; amsacrine; anastrozole; anthramycin; asparaginase; asperlin; azacitidine; azetepa; azotomycin; batimastat; benzodepa; bicalutamide; bisantrene hydrochloride; bisnafide dimesylate; bizelesin; bleomycin sulfate; brequinar sodium; bropirimine; busulfan; cactinomycin; calusterone; caracemide; carbetimer; carboplatin; carmustine; carubicin hydrochloride; carzelesin; cedefingol; chlorambucil; cirolemycin; cladribine; crisnatol mesylate; cyclophosphamide; cytarabine; dacarbazine; daunorubicin hydrochloride; decitabine; dexormaplatin; dezaguanine; dezaguanine mesylate; diaziquone; doxorubicin; doxorubicin hydrochloride; droloxifene; droloxifene citrate; dromostanolone propionate; duazomycin; edatrexate; eflornithine hydrochloride; elsamitrucin; enloplatin; enpromate; epipropidine; epirubicin hydrochloride; erbulozole; esorubicin hydrochloride; estramustine; estramustine phosphate sodium; etanidazole; etoposide; etoposide phosphate; etoprine; fadrozole hydrochloride; fazarabine; fenretinide; floxuridine; fludarabine phosphate; fluorouracil; flurocitabine; fosquidone; fostriecin sodium; gemcitabine; gemcitabine hydrochloride; hydroxyurea; idarubicin hydrochloride; ifosfamide; ilmofosine; interleukin II (including recombinant interleukin II, or rIL2), interferon alfa-2a; interferon alfa-2b; interferon alfa-n1; interferon alfa-n3; interferon beta-I a; interferon gamma-I b; iproplatin; irinotecan hydrochloride; lanreotide acetate; letrozole; leuprolide acetate; liarozole hydrochloride; lometrexol sodium; lomustine; losoxantrone hydrochloride; masoprocol; maytansine; mechlorethamine hydrochloride; megestrol acetate; melengestrol acetate; melphalan; menogaril; mercaptopurine; methotrexate; methotrexate sodium; metoprine; meturedepa; mitindomide; mitocarcin; mitocromin; mitogillin; mitomalcin; mitomycin; mitosper; mitotane; mitoxantrone hydrochloride; mycophenolic acid; nocodazole; nogalamycin; ormaplatin; oxisuran; pegaspargase; peliomycin; pentamustine; peplomycin sulfate; perfosfamide; pipobroman; piposulfan; piroxantrone hydrochloride; plicamycin; plomestane; porfimer sodium; porfiromycin; prednimustine; procarbazine hydrochloride; puromycin; puromycin hydrochloride; pyrazofurin; riboprine; rogletimide; safingol; safingol hydrochloride; semustine; simtrazene; sparfosate sodium; sparsomycin; spirogermanium hydrochloride; spiromustine; spiroplatin; streptonigrin; streptozocin; sulofenur; talisomycin; tecogalan sodium; tegafur; teloxantrone hydrochloride; temoporfin; teniposide; teroxirone; testolactone; thiamiprine; thioguanine; thiotepa; tiazofurin; tirapazamine; toremifene citrate; trestolone acetate; triciribine phosphate; trimetrexate; trimetrexate glucuronate; triptorelin; tubulozole hydrochloride; uracil mustard; uredepa; vapreotide; verteporfin; vinblastine sulfate; vincristine sulfate; vindesine; vindesine sulfate; vinepidine sulfate; vinglycinate sulfate; vinleurosine sulfate; vinorelbine tartrate; vinrosidine sulfate; vinzolidine sulfate; vorozole; zeniplatin; zinostatin; zorubicin hydrochloride.

Other anti-cancer drugs include, but are not limited to: 20-epi-1,25 dihydroxyvitamin D3; 5-ethynyluracil; abiraterone; aclarubicin; acylfulvene; adecypenol; adozelesin; aldesleukin; ALL-TK antagonists; altretamine; ambamustine; amidox; amifostine; aminolevulinic acid; amrubicin; amsacrine; anagrelide; anastrozole; andrographolide; angiogenesis inhibitors; antagonist D; antagonist G; antarelix; anti-dorsalizing morphogenetic protein-1; antiandrogen, prostatic carcinoma; antiestrogen; antineoplaston; antisense oligonucleotides; aphidicolin glycinate; apoptosis gene modulators; apoptosis regulators; apurinic acid; ara-CDP-DL-PTBA; arginine deaminase; asulacrine; atamestane; atrimustine; axinastatin 1; axinastatin 2; axinastatin 3; azasetron; azatoxin; azatyrosine; baccatin III derivatives; balanol; batimastat; BCR/ABL antagonists; benzochlorins; benzoylstaurosporine; beta lactam derivatives; beta-alethine; betaclamycin B; betulinic acid; bFGF inhibitor; bicalutamide; bisantrene; bisaziridinylspermine; bisnafide; bistratene A; bizelesin; breflate; bropirimine; budotitane; buthionine sulfoximine; calcipotriol; calphostin C; camptothecin derivatives; canarypox IL-2; capecitabine; carboxamide-amino-triazole; carboxyamidotriazole; CaRest M3; CARN 700; cartilage derived inhibitor; carzelesin; casein kinase inhibitors (ICOS); castanospermine; cecropin B; cetrorelix; chlorlns; chloroquinoxaline sulfonamide; cicaprost; cis-porphyrin; cladribine; clomifene analogues; clotrimazole; collismycin A; collismycin B; combretastatin A4; combretastatin analogue; conagenin; crambescidin 816; crisnatol; cryptophycin 8; cryptophycin A derivatives; curacin A; cyclopentanthraquinones; cycloplatam; cypemycin; cytarabine ocfosfate; cytolytic factor; cytostatin; dacliximab; decitabine; dehydrodidenmin B; deslorelin; dexamethasone; dexifosfamide; dexrazoxane; dexverapamil; diaziquone; didemnin B; didox; diethylnorspermine; dihydro-5-azacytidine; 9-dioxamycin; diphenyl spiromustine; docosanol; dolasetron; doxifluridine; droloxifene; dronabinol; duocarmycin SA; ebselen; ecomustine; edelfosine; edrecolomab; eflornithine; elemene; emitefur; epirubicin; epristeride; estramustine analogue; estrogen agonists; estrogen antagonists; etanidazole; etoposide phosphate; exemestane; fadrozole; fazarabine; fenretinide; filgrastim; finasteride; flavopiridol; flezelastine; fluasterone; fludarabine; fluorodaunorunicin hydrochloride; forfenimex; formestane; fostriecin; fotemustine; gadolinium texaphyrin; gallium nitrate; galocitabine; ganirelix; gelatinase inhibitors; gemcitabine; glutathione inhibitors; hepsulfam; heregulin; hexamethylene bisacetamide; hypericin; ibandronic acid; idarubicin; idoxifene; idramantone; ilmofosine; ilomastat; imidazoacridones; imiquimod; immunostimulant peptides; insulin-like growth factor-1 receptor inhibitor; interferon agonists; interferons; interleukins; iobenguane; iododoxorubicin; ipomeanol, 4-; iroplact; irsogladine; isobengazole; isohomohalicondrin B; itasetron; jasplakinolide; kahalalide F; lamellarin-N triacetate; lanreotide; leinamycin; lenograstim; lentinan sulfate; leptolstatin; letrozole; leukemia inhibiting factor; leukocyte alpha interferon; leuprolide+estrogen+progesterone; leuprorelin; levamisole; liarozole; linear polyamine analogue; lipophilic disaccharide peptide; lipophilic platinum compounds; lissoclinamide 7; lobaplatin; lombricine; lometrexol; lonidamine; losoxantrone; lovastatin; loxoribine; lurtotecan; lutetium texaphyrin; lysofylline; lytic peptides; maitansine; mannostatin A; marimastat; masoprocol; maspin; matrilysin inhibitors; matrix metalloproteinase inhibitors; menogaril; merbarone; meterelin; methioninase; metoclopramide; MIF inhibitor; mifepristone; miltefosine; mirimostim; mismatched double stranded RNA; mitoguazone; mitolactol; mitomycin analogues; mitonafide; mitotoxin fibroblast growth factor-saporin; mitoxantrone; mofarotene; molgramostim; monoclonal antibody, human chorionic gonadotrophin; monophosphoryl lipid A+myobacterium cell wall sk; mopidamol; multiple drug resistance gene inhibitor; multiple tumor suppressor 1-based therapy; mustard anticancer agent; mycaperoxide B; mycobacterial cell wall extract; myriaporone; N-acetyldinaline; N-substituted benzamides; nafarelin; nagrestip; naloxone+pentazocine; napavin; naphterpin; nartograstim; nedaplatin; nemorubicin; neridronic acid; neutral endopeptidase; nilutamide; nisamycin; nitric oxide modulators; nitroxide antioxidant; nitrullyn; O6-benzylguanine; octreotide; okicenone; oligonucleotides; onapristone; ondansetron; ondansetron; oracin; oral cytokine inducer; ormaplatin; osaterone; oxaliplatin; oxaunomycin; palauamine; palmitoylrhizoxin; pamidronic acid; panaxytriol; panomifene; parabactin; pazelliptine; pegaspargase; peldesine; pentosan polysulfate sodium; pentostatin; pentrozole; perflubron; perfosfamide; perillyl alcohol; phenazinomycin; phenylacetate; phosphatase inhibitors; picibanil; pilocarpine hydrochloride; pirarubicin; piritrexim; placetin A; placetin B; plasminogen activator inhibitor; platinum complex; platinum compounds; platinum-triamine complex; porfimer sodium; porfiromycin; prednisone; propyl bis-acridone; prostaglandin J2; proteasome inhibitors; protein A-based immune modulator; protein kinase C inhibitor; protein kinase C inhibitors, microalgal; protein tyrosine phosphatase inhibitors; purine nucleoside phosphorylase inhibitors; purpurins; pyrazoloacridine; pyridoxylated hemoglobin polyoxyethylene conjugate; raf antagonists; raltitrexed; ramosetron; ras farnesyl protein transferase inhibitors; ras inhibitors; ras-GAP inhibitor; retelliptine demethylated; rhenium Re 186 etidronate; rhizoxin; ribozymes; RII retinamide; rogletimide; rohitukine; romurtide; roquinimex; rubiginone B1; ruboxyl; safingol; saintopin; SarCNU; sarcophytol A; sargramostim; Sdi 1 mimetics; semustine; senescence derived inhibitor 1; sense oligonucleotides; signal transduction inhibitors; signal transduction modulators; single chain antigen-binding protein; sizofiran; sobuzoxane; sodium borocaptate; sodium phenylacetate; solverol; somatomedin binding protein; sonermin; sparfosic acid; spicamycin D; spiromustine; splenopentin; spongistatin 1; squalamine; stem cell inhibitor; stem-cell division inhibitors; stipiamide; stromelysin inhibitors; sulfinosine; superactive vasoactive intestinal peptide antagonist; suradista; suramin; swainsonine; synthetic glycosaminoglycans; tallimustine; tamoxifen methiodide; tauromustine; tazarotene; tecogalan sodium; tegafur; tellurapyrylium; telomerase inhibitors; temoporfin; temozolomide; teniposide; tetrachlorodecaoxide; tetrazomine; thaliblastine; thiocoraline; thrombopoietin; thrombopoietin mimetic; thymalfasin; thymopoietin receptor agonist; thymotrinan; thyroid stimulating hormone; tin ethyl etiopurpurin; tirapazamine; titanocene bichloride; topsentin; toremifene; totipotent stem cell factor; translation inhibitors; tretinoin; triacetyluridine; triciribine; trimetrexate; triptorelin; tropisetron; turosteride; tyrosine kinase inhibitors; tyrphostins; UBC inhibitors; ubenimex; urogenital sinus-derived growth inhibitory factor; urokinase receptor antagonists; vapreotide; variolin B; vector system, erythrocyte gene therapy; velaresol; veramine; verdins; verteporfin; vinorelbine; vinxaltine; vitaxin; vorozole; zanoterone; zeniplatin; zilascorb; and zinostatin stimalamer. Preferred additional anti-cancer drugs are 5-fluorouracil and leucovorin.

Examples of therapeutic antibodies that can be used as anti-cancer agents include but are not limited to HERCEPTIN® (Trastuzumab) (Genentech, CA) which is a humanized anti-HER2 monoclonal antibody for the treatment of patients with metastatic breast cancer; REOPRO® (abciximab) (Centocor) which is an anti-glycoprotein IIb/IIIa receptor on the platelets for the prevention of clot formation; ZENAPAX® (daclizumab) (Roche Pharmaceuticals, Switzerland) which is an immunosuppressive, humanized anti-CD25 monoclonal antibody for the prevention of acute renal allograft rejection; PANOREX™ which is a murine anti-17-IA cell surface antigen IgG2a antibody (Glaxo Wellcome/Centocor); BEC2 which is a murine anti-idiotype (GD3 epitope) IgG antibody (ImClone System); IMC-C225 which is a chimeric anti-EGFR IgG antibody (ImClone System); VITAXIN™ which is a humanized anti-αVβ3 integrin antibody (Applied Molecular Evolution/MedImmune); Campath 1H/LDP-03 which is a humanized anti CD52 IgG1 antibody (Leukosite); Smart M195 which is a humanized anti-CD33 IgG antibody (Protein Design Lab/Kanebo); RITUXAN™ which is a chimeric anti-CD20 IgG1 antibody (IDEC Pharm/Genentech, Roche/Zettyaku); LYMPHOCIDE™ which is a humanized anti-CD22 IgG antibody (Immunomedics); LYMPHOCIDE™ Y-90 (Immunomedics); Lymphoscan (Tc-99m-labeled; radioimaging; Immunomedics); Nuvion (against CD3; Protein Design Labs); CM3 is a humanized anti-ICAM3 antibody (ICOS Pharm); IDEC-114 is a primatied anti-CD80 antibody (IDEC Pharm/Mitsubishi); ZEVALIN™ is a radiolabelled murine anti-CD20 antibody (IDEC/Schering AG); IDEC-131 is a humanized anti-CD40L antibody (IDEC/Eisai); IDEC-151 is a primatized anti-CD4 antibody (IDEC); IDEC-152 is a primatized anti-CD23 antibody (IDEC/Seikagaku); SMART anti-CD3 is a humanized anti-CD3 IgG (Protein Design Lab); 5G1.1 is a humanized anti-complement factor 5 (C5) antibody (Alexion Pharm); D2E7 is a humanized anti-TNF-α antibody (CAT/BASF); CDP870 is a humanized anti-TNF-α Fab fragment (Celltech); IDEC-151 is a primatized anti-CD4 IgG1 antibody (IDEC Pharm/SmithKline Beecham); MDX-CD4 is a human anti-CD4 IgG antibody (Medarex/Eisai/Genmab); CD20-sreptdavidin (+biotin-yttrium 90; NeoRx); CDP571 is a humanized anti-TNF-α IgG4 antibody (Celltech); LDP-02 is a humanized anti-α4β7 antibody (LeukoSite/Genentech); OrthoClone OKT4A is a humanized anti-CD4 IgG antibody (Ortho Biotech); ANTOVA™ is a humanized anti-CD40L IgG antibody (Biogen); ANTEGREN™ is a humanized anti-VLA-4 IgG antibody (Elan); and CAT-152 is a human anti-TGF-β₂ antibody (Cambridge Ab Tech).

Chemotherapeutic agents that can be used in the methods and compositions of the invention include but are not limited to alkylating agents, antimetabolites, natural products, or hormones. Examples of alkylating agents useful for the treatment or prevention of T-cell malignancies in the methods and compositions of the invention include but are not limited to, nitrogen mustards (e.g., mechloroethamine, cyclophosphamide, chlorambucil, etc.), alkyl sulfonates (e.g., busulfan), nitrosoureas (e.g., carmustine, lomusitne, etc.), or triazenes (decarbazine, etc.). Examples of antimetabolites useful for the treatment or prevention of T-cell malignancies in the methods and compositions of the invention include but are not limited to folic acid analog (e.g., methotrexate), or pyrimidine analogs (e.g., Cytarabine), purine analogs (e.g., mercaptopurine, thioguanine, pentostatin). Examples of natural products useful for the treatment or prevention of T-cell malignancies in the methods and compositions of the invention include but are not limited to vinca alkaloids (e.g., vinblastin, vincristine), epipodophyllotoxins (e.g., etoposide), antibiotics (e.g., daunorubicin, doxorubicin, bleomycin), enzymes (e.g., L-asparaginase), or biological response modifiers (e.g., interferon alpha).

Examples of alkylating agents useful for the treatment or prevention of cancer as anti-cancer agents in the methods and compositions of the invention include but are not limited to, nitrogen mustards (e.g., mechloroethamine, cyclophosphamide, chlorambucil, melphalan, etc.), ethylenimine and methylmelamines (e.g., hexamethlymelamine, thiotepa), alkyl sulfonates (e.g., busulfan), nitrosoureas (e.g., carmustine, lomusitne, semustine, streptozocin, etc.), or triazenes (decarbazine, etc.). Examples of antimetabolites useful for the treatment or prevention of cancer in the methods and compositions of the invention include but are not limited to folic acid analog (e.g., methotrexate), or pyrimidine analogs (e.g., fluorouracil, floxouridine, Cytarabine), purine analogs (e.g., mercaptopurine, thioguanine, pentostatin). Examples of natural products useful for the treatment or prevention of cancer in the methods and compositions of the invention include but are not limited to vinca alkaloids (e.g., vinblastin, vincristine), epipodophyllotoxins (e.g., etoposide, teniposide), antibiotics (e.g., actinomycin D, daunorubicin, doxorubicin, bleomycin, plicamycin, mitomycin), enzymes (e.g., L-asparaginase), or biological response modifiers (e.g., interferon alpha). Examples of hormones and antagonists useful for the treatment or prevention of cancer in the methods and compositions of the invention include but are not limited to adrenocorticosteroids (e.g., prednisone), progestins (e.g., hydroxyprogesterone caproate, megestrol acetate, medroxyprogesterone acetate), estrogens (e.g., diethlystilbestrol, ethinyl estradiol), antiestrogen (e.g., tamoxifen), androgens (e.g., testosterone propionate, fluoxymesterone), antiandrogen (e.g., flutamide), gonadotropin releasing hormone analog (e.g., leuprolide). Other agents that can be used in the methods and compositions of the invention for the treatment or prevention of cancer include platinum coordination complexes (e.g., cisplatin, carboblatin), anthracenedione (e.g., mitoxantrone), substituted urea (e.g., hydroxyurea), methyl hydrazine derivative (e.g., procarbazine), adrenocortical suppressant (e.g., mitotane, aminoglutethimide).

A “subject” is a mammal, preferably a human, but can also be an animal in need of veterinary treatment, e.g., companion animals (e.g., dogs, cats, and the like), farm animals (e.g., cows, sheep, pigs, horses, and the like) and laboratory animals (e.g., rats, mice, guinea pigs, and the like).

The present invention is illustrated by the following examples, which are not intended to be limiting in any way.

### EXEMPLIFICATION

### Procedures for Examples 1-4

Compound (1) was suspended in 1% methylcellulose solution (Wako, Osaka, 400 c.p., and distilled water) and was stored at 4° C. Paclitaxel (Sigma St. Louis, Mo.) was dissolved in dimethylsulfoxide (DMSO, Nakarai-Tesk, Tokyo) and stored at 4° C., and was prepared in a solution of 10% DMSO, 18% Cremophore RH-40 (Nikko Chemicals) and 72% distilled water before use.

Female mice, 8 weeks old (strain BALB/c-nu/nu, CLEA Japan Inc., Tokyo) were obtained and adapted for 5 days at experimental conditions and used at 10 weeks old. Experimental conditions included temperature of 21-27° C.; relative humidity of 40-70%; ventilation>10 times/hour; fluorescent light for 12-hour light (8:00-20:00)/12-hour dark; ad libitium access to food (irradiated CA-1, CLEA Japan Inc., Tokyo) and water (Osaka city tap water).

Statistical analyses versus control group and between selected groups were performed using a spreadsheet program to run Welch's test and Dunnett's multiple comparison test.

Tumor size and body weight were scored at least twice a week throughout the experiment. Tumor size was captured utilizing CD-10C compatible electric calipers (Mitsutoyo Corp., Kawasaki) and animal weight was automatically captured from the balance (A/D, Tokyo). The tumor volume was calculated by the following formula: [Tumor volume=(Length×Width×Width)/2]. The relative tumor volume (RV) was calculated as RV=Vn/Vo, where Vn: tumor volume on day n, and Vo: initial tumor volume. Judgment was performed one day (Examples 6 and 9) or three days (Examples 7 and 8) after the last dosing.

Animal experiments were performed using Tumor Measuring & Tracking System (VISIONS Co. Ltd., Tokyo). An experiment consisted of 8 mice per group (Examples 6 and 9) or 6 mice per group (Examples 7 and 8). Tumor samples were implanted subcutaneously into the back of mice (day 0). Tumor-implanted animals were randomized to groups based upon tumor volume and body weight when tumor volume reached approximately 100-200 mm³. The control group was given vehicle (1% methylcellulose solution). The doses were calculated on the basis of up-to-date body weights of the mice (0.1 mL/10 g body weight). The mice were non-fasted during an entire course of the experiment.

### Example 1

### Compound (1) Is Effective Against Human Squamous Cell Carcinoma

Ma44 human lung squamous cell carcinoma was maintained by in vitro passage with using Eagle's Minimum Essential Medium (Nissui Pharmaceutical Co., Tokyo) supplemented with 10% fetal calf serum (Life Technologies Inc, MD). Ma44 cells (5×10⁵/0.1 mL) were implanted and the animals were randomized at 11 days. Compound (1) was orally administered daily for 14 days. Paclitaxel solution (DMSO/Cremophore/distilled water) was prepared as above and administered intravenously 6 times (3 times per week×2).

Table 1 summarizes the following results. The antitumor efficacy of Compound (1) against Ma44 human lung cancer was evaluated and compared with Paclitaxel. Daily oral dosing of Compound (1) at 50 mg/kg showed significant (p<0.01) and effective (>50% inhibition) growth inhibition against Ma44 cells (63.9% inhibition). While body weight losses were mild, one of eight mice was found dead, suggesting that 50 mg/kg is close to maximum tolerated dose in this schedule. Thus, data from this mouse is not provided on day 24. A lower dose of Compound (1) resulted in significant (p<.0.01) but less effective growth inhibition (36.2% inhibition).

Antitumor activity of Taxol at a dose of 20 mg/kg, the maximum tolerated dose for nude mice in this dosing schedule, was neither significant nor effective against Ma44 cells. These results demonstrated that Compound (1) showed superior antitumor activity than that of taxol.

TABLE 1
Compound (1) Is Effective Against Human Squamous Cell Carcinoma

| | | No. | | | |
|---|---|---|---|---|---|
| | Total dose | of | RV^{a)} on day 27 | % | MBWL |
| Compound | (mg/kg) | mice | (Mean +/− SD) | Inhibition | (%)^{b)} |
| Vehicle | 0 | 6 | 15.4 ± 4.6 | | 3 |
| Compound (1) | 25 × 14^{c)} | 6 | 10.0 ± 1.4* | 35.2 | 3 |
| Compound (1) | 50 × 14 | 6 | 4.8 ± 1.7** | 68.8 | 5 |
| Compound (1) | 75 × 14 | 6 | Toxic death on | | |
| | | | day 18-22 | | |
| Paclitaxel | 20 × 6^{d)} | 6 | 2.8 ± 0.4^{**e)} | 81.8 | 3 |

^{a)}Relative tumor volume
^{b)}maximum body weight loss (% of initial)
^{c)}day 11-24, d): day 11, 13, 15, 18, 20 and 22
^{e)}p < 0.01 for group No. 69 and 72 by Dunnett's test
*,**p < 0.05, 0.01 for vehicle control (group No. 68) by Welch's test

### Example 2

### Compound (1) Is Effective Against Human Lung Cancer (RERF-LC-AI Carcinoma)

The tumor sample implanted was a fragment of RERF-LC-AI carcinoma. Implanted animals were randomized at 10 days. Compound (1) was orally administered daily for 14 days. Paclitaxel solution (DMSO/Cremophore/distilled water) was prepared as above. and administered intravenously 6 times (3 times per week×2, day 10, 12, 14, 17, 19 and 21).

Table 2 summarizes the following results. The antitumor efficacy of Compound (1) against RERF.LC-AI human lung cancer was evaluated and compared with that of Paclitaxel. Daily oral dosing of Compound (1) at 50 mg/kg and 25 mg/kg showed significant (p<0.05) and effective (>50% inhibition) growth inhibition against RERF-LC.AI cells (79.7% inhibition at 50 mg/kg and 64.9% inhibition at 25 mg/kg). However, one of six mice given Compound (1) at 50 mg/kg was found dead, suggesting that 50 mg/kg is close to maximum tolerated dose in this schedule. All mice given 75 mg/kg Compound (1) were found dead, indicating this dose was toxic. Therefore the data from these mice were not available on the date for evaluation of day 26.

Treatment with Compound (1) was effective, showing potent antitumor inhibition at 25 mg/kg (64.9%) and 50 mg/kg (79.7%). However, Paclitaxel at a dose of 20 mg/kg, the maximum tolerated dose for nude mice in this dosing schedule, also showed potent antitumor effect against RERF.LC-AI cells with significant (p<0.05) growth inhibition of 92.2%, which was greater than that of Compound (1).

TABLE 2
Compound (1) Is Effective Against Human Lung Cancer (RERF-LC-AI
Carcinoma)

| | | No. | | | |
|---|---|---|---|---|---|
| | Total dose | of | RV^{a)} on day 26 | % | MBWL |
| Compound | (mg/kg) | mice | (Mean +/− SD) | Inhibition | (%)^{b)} |
| Vehicle | 0 | 6 | 40.3 ± 14.5 | | 2 |
| Compound (1) | 25 × 14^{c)} | 6 | 14.1 ± 73** | 64.9 | 4 |
| Compound (1) | 50 × 14 | 5 | 8.2 ± 1.9** | 79.7 | 14 |
| | | 1 | Toxic death on | | 32 |
| | | | day 21 | | |
| Compound (1) | 75 × 14 | 6 | Toxic death on | | 20 |
| | | | day 17-20 | | |
| Paclitaxel | 20 × 6^{d)} | 7 | 3.1 ± 0.8**^{e)} | 92.2 | 7 |

^{a)}Relative tumor volume
^{b)}maximum body weight loss (% of initial)
^{c)}day 10-23, d) day 10, 12, 14, 17, 19 and 21
^{d)}p < 0.O5 for group No. 1230, 1233 and 1234 by Dunnett's test ‘p < 0.05, 0.01 for vehicle control (group No. 1229) by Welch's test

### Example 3

### Compound (1) Is Effective Against Human Lung Cancer (RERF-LC-AI/TXL Carcinoma)

The tumor sample implanted was a fragment of RERF-LC-AI/TXL carcinoma. Implanted animals were randomized at 11 days. Compound (1) was orally administered daily for 14 days. Paclitaxel solution (DMSO/Cremophore/distilled water) was prepared as above. and administered intravenously 6 times (3 times per week×2, day 11, 13, 15, 18, 20 and 22).

Table 3 summarizes the following results. The antitumor efficacy of Compound (1) against RERF-LC-AI/TXL human lung cancer was evaluated and compared with that of Paclitaxel. Daily oral dosing of Compound (1) at 50 mg/kg and 25 mg/kg showed significant (p<0.01 and 0.05) growth inhibition against RERF-LC-AI/TXL cells. Treatment with Compound (1) at 50 mg/kg resulted in effective (>50% inhibition) antitumor efficacy with 68.8% growth inhibition. All mice given 75 mg/kg Compound (1) were found dead, suggesting this dose was toxic. Thus, the data from these mice were not available on the date for evaluation of day 27.

Treatment with Paclitaxel at a dose of 20 mg/kg, a maximum tolerated close for nude mice in this dosing schedule, showed potent antitumor effect against RERF-LC-AI/TXL cells with growth inhibition of 81.8%. The sensitivity of RERF/LC-AI/TXL to Paclitaxel is lower than that of RFRF-LC-AI, parental cell line of RERF/LC-AI/TXL, but it looks higher than usual in this study. Antitumor effect of Paclitaxel in this study is statistically equivalent to Compound (1).

TABLE 3
Compound (1) Is Effective Against Human Lung Cancer
(RERF-LC-AI/TXL Carcinoma)

| | No. | | | | |
|---|---|---|---|---|---|
| | of | Total dose | RV^{a)} on day 24 | % | MBWL |
| Compound | mice | (mg/kg) | (Mean +/− SD) | inhibition | (%)^{b)} |
| vehicle | 8 | 0 | 41.56 ± 13.31 | | 0 |
| Compound (1) | 8 | 25 × 14c) | 26.49 ± 13.41* | 36.2 | 1 |
| Compound (1) | 7 | 50 × 14 | 15.01 ± 6.45** | 63.9 | 3 |
| | 1 | Toxic | | | |
| | | death on | | | |
| | | day 16 | | | |
| Paclitaxel | 8 | 10 × 6^{d)} | 31.23 ± 6.69 | 24.8 | 0 |
| Paclitaxel | 8 | 20 × 6^{d)} | 32.87 ± 10.03 | 20.9 | 0 |

^{a)}Relative tumor volume
^{b)}maximum body weight loss (% of initial)
^{c)}from day 10 to 23, d): day 10, 12, 14, 17, 19 and 21
*,**p < 0.05, 0.01 for vehicle control (group No. 581) by Welch's test
#: p < 0.01 for Paclitaxel alone (group 586)

### Example 4

### Compound (1) Is Superior to Taxol in Effectiveness Against Human Ovarian Cancer

The tumor sample implanted included BG-1-TR1 cells (3×10⁶/0.1 ml of 5 mg/ml matrigel solution, BD Biosciences, Bedford, Mass.). Implanted animals were randomized at 20 days. Compound (1) was orally administered daily for 14 days from day 20 to 33. Paclitaxel solution (DMSO/Cremophore/distilled water) was prepared as above. and administered intravenously 6 times (day 20, 22, 24, 27, 29 and 31).

Table 4 summarizes the following results. The antitumor efficacy of Compound (1) against BG-1-TR1 human ovarian cancer was evaluated and compared with that of Paclitaxel. Daily oral dosing of Compound (1) at 50 mg/kg showed significant (p<0.01) and effective (>50% inhibition) growth inhibition against BG-1-TR1 human ovarian cancer (6 1.4% inhibition). Lower dose of Compound (1) at 25 mg/kg resulted in significant (p<0.05) but not effective growth inhibition (27.5% inhibition). The body weight losses in these groups were found to be mild.

The antitumor activity of Taxol at a dose of 20 mg/kg, a maximum tolerated dose for nude mice in this dosing schedule, was neither significant nor effective against BG-1 cells, since these cells were known to be highly resistant to Taxol.

The antitumor efficacy of Compound (1) was significantly (p<0.01) superior to that of Taxol. These results demonstrated that Compound (1) was effective against Taxol human ovarian cancer.

TABLE 4
Compound (1) Is Superior to Taxol in Effectiveness Against Human Ovarian Cancer

| | No. of | Total dose | | | |
|---|---|---|---|---|---|
| Compound | mice | (mg/kg) | RV^{a)} (Mean +/− SD) | % inhibition | MBWL (%)^{d)} |
| vehicle | 8 | 0 | 2.2 ± 0.6 | 0 | 0.7% (d27) |
| Paclitaxel | 8 | 20 mg/kg × 6^{a)} | 2.0 ± 0.7 | 8.3 | 4.5% (d27) |
| Compound (1) | 8 | 12.5 mg/kg × 14^{b)} | 1.6 ± 0.7 | 27.4 | 0 |
| Compound (1) | 8 | 25 mg/kg × 14^{b)} | 1.6 ± 0.5* | 27.5 | 1.9% (d24) |
| Compound (1) | 8 | 50 mg/kg × 14^{b)} | 0.9 ± 0.3 | 61.4 | 8.7% (d34) |

^{a)}On day 20, 22, 24, 27, 29, 31
^{b)}from day 20 to 33
^{c)}Relative tumor volume (mean ± SD) on day 34
^{d)}Maximum body weight loss (% of initial)
*,**p < 0.05, 0.01 when compared to group 35 by welch's t-test

While this invention has been particularly shown and described with references to preferred embodiments thereof, it will be understood by those skilled in the art that various changes in form and details may be made therein without departing from the scope of the invention encompassed by the appended claims.

1. A method of treating a subject having lung cancer or ovarian cancer, comprising administering to the subject an effective amount of a compound represented by the following structural formula: 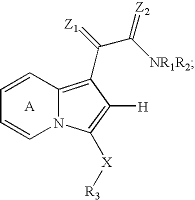
or a pharmaceutically acceptable salt, solvate or polymorph thereof, wherein:
Ring A is substituted or unsubstituted and is optionally fused to an aryl group;
Z₁ and Z₂ are independently ═O, ═S, ═N—OR₁₂ or ═NR₁₂.
R₁ and R₂ are independently —H, an aliphatic group, a substituted aliphatic group, an unsubstituted non-aromatic heterocylic group, a substituted non-aromatic heterocylic group, an unsubstituted aryl group or a substituted aryl group, provided that R₁ and R₂ are not both —H; or —NR₁R_{2,} taken together, is a substituted or unsubstituted non-aromatic nitrogen-containing heterocyclic group or a substituted or unsubstituted nitrogen-containing heteroaryl group;
R₃ is a substituted or unsubstituted aryl group or a substituted or unsubstituted aliphatic group;
X is a covalent bond, —C(R₄R₅)—, —N(R₄)—, —O—, —S—, —S(O)—, —S(O)₂—, —C(═O)—, —C(═O)—N(R₄)—, or —N(R₄)—C(═O)—;
R₄ and R₅ are independently —H or a substituted or unsubstituted aliphatic group; and
R₁₂ is —H or a substituted or unsubstituted alkyl group.

2. The method of claim 1 wherein the subject has human ovarian cancer.

3. The method of claim 2 wherein the subject has epithelial ovarian carcinoma.

4. The method of claim 2 wherein the subject has ovarian germ cell carcinoma.

5. The method of claim 2 wherein the subject has ovarian stromal carcinoma.

6. The method of claim 1 wherein the subject has human primary lung cancer.

7. The method of claim 6 wherein the subject has squamous cell carcinoma.

8. The method of claim 6 wherein the subject has small cell carcinoma.

9. The method of claim 6 wherein the subject has adenocarcinoma.

10. The method of claim 6 wherein the subject has large cell carcinoma.

11. The method of claim 1 wherein: Ring A substituted or unsubstituted, Z₁ and Z₂ are both ═O; R₁ is —H; R₂ is a substituted or unsubstituted alkyl or aryl group; R₃ is a substituted or unsubstituted aryl group; and X is —C(R₄R₅)—, —N(R₄)— or —O—.

12. The method of claim 11 wherein R₂ is represented by a structural formula selected from: 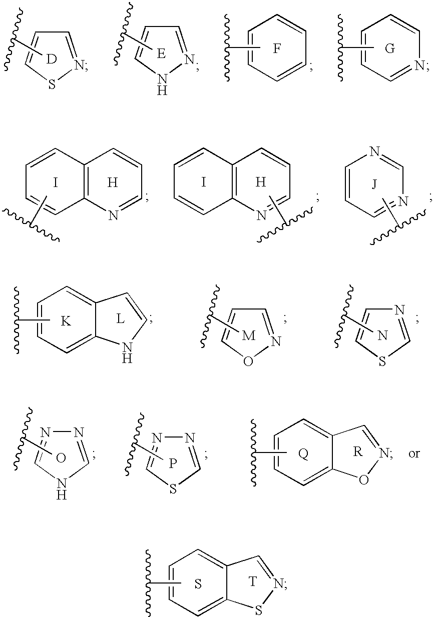
wherein Rings D-T are substituted or unsubstituted.

13. The method of claim 12 wherein zero, one or more ring carbons atoms of Rings D-T are substituted with a group independently selected from —OH, —Br, —Cl, —I, —F, —OR^{a}, —O—COR^{a}, —COR^{a}, —CN, —NO₂, —COOH, —SO₃H, —NH₂, —NHR^{a}, —N(R^{a}R^{b}), —COOR^{a}, —CHO, —CONH₂, —CONHR^{a}, —CON(R^{a}R^{b}), —NHCOR^{a}, —NRCOR^{a}, —NHCONH₂, —NHCONR^{a}H, —NHCON(R^{a}R^{b}), —NRCCONH₂, —NR^{c}CONR^{a}H, —NR^{c}CON(R^{a}R^{b}), —C(═NH)—NH₂, —C(═NH)—NHR^{a}, —C(═NH)—N(R^{a}R^{b}), —C(═NR^{c})—NH₂, —C(═NR^{c})—NHR^{a}, —C(═NR^{c})—N(R^{a}R^{b}), —NH—C(═NH)—NH₂, —NH—C(═NH)—NHR^{a}, —NH—C(═NH)—N(R^{a}R^{b}), —NH—C(═NR^{c})—NH₂, —NH—C(═NR^{c})—NHR^{a}, —NH—C(═NR^{c})—N(R^{a}R^{b}), —NR^{d}H-C(═NH)—NH₂, —NR^{d}—C(═NH)—NHR^{a}, —NR^{d}—C(═NH)—N(R^{a}R^{b}), —NR^{d}—C(═NR^{c})—NH₂, —NR^{d}—C(═NR^{c})—NHR^{a}, —NR^{d}—C(═NR^{c})—N(R^{a}R^{b}), —NHNH₂, —NHNHR^{a}, —NHR^{a}R^{b}, —SO₂NH₂, —SO₂NHR^{a}, —SO₂NR^{a}R^{b}, —CH═CHR^{a}, —CH═CR^{a}R^{b}, —CR^{c}═CR^{a}R^{b}, —CR^{c}═CHR^{a}, —CR^{c}═CR^{a}R^{b}, —CCR^{a}, —SH, —SR^{a}, —S(O)R^{a}, —S(O)₂R^{a}, alkyl groups, substituted alkyl group, non-aromatic heterocyclic group, substituted non-aromatic heterocyclic group, benzyl group, substituted benzyl group, aryl group or substituted aryl group wherein R^{a}-R^{d} are each independently an alkyl group, substituted alkyl group, benzyl, substituted benzyl, aryl or substituted aryl group, or, —N(R^{a}R^{b}), taken together, can also form a substituted or unsubstituted non-aromatic heterocyclic group.

14. The method of claim 12 wherein zero one or more ring carbon atoms of Rings D-T are independently substituted with a group selected from C1-C4 alkyl, C1-C4 hydroxyalkyl, N-morpholino, pyrimidyl, C1-C4 alkyl substituted pyrimidyl, —NH(C1-C4 alkyl), —N(C1-C4 alkyl)₂, —C(O)NH₂, —C(O)NH(C1-C4 alkyl), C(O)N(C1-C4 alkyl)₂, —NHC(O)(C1-C4 alkyl), —NO₂, C1-C4 alkoxy, —C(O)O—CH₂CH₂—NH(C1-C4 alkyl), —C(O)O—CH₂CH₂—N(C1-C4 alkyl)₂, 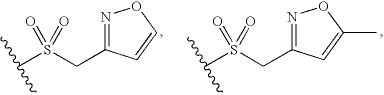
—NH-(phenyl), —NH₂, —CH₂NH—C(O)—O—(C1-C4 alkyl), —CH₂NH₂, —Cl, —F, —C(O)—O—(C1-C4 alkyl), —C(O)—NH—(C1-C4 alkyl), C3-C7 cycloalkyl, phenyl, —C(O)—N-morpholino, —S—(C1-C4 alkyl), —CN, furyl, —S(O)₂—(C1-C4 alkyl), —S(O)₂—NH₂, —S(O)₂—NH(C1-C4 alkyl), —S(O)₂—N(C1-C4 alkyl)₂.

15. The method of claim 14 wherein R₂ is represented by a structural formula selected from: 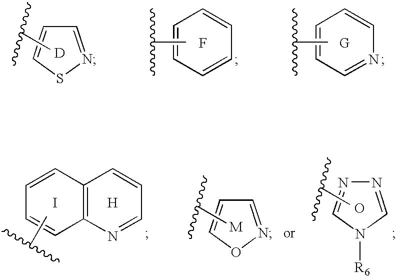
and R₆ is —H or a substituted or unsubstituted alkyl group

16. The method of claim 15 wherein R₂ is represented by a structural formula selected from: 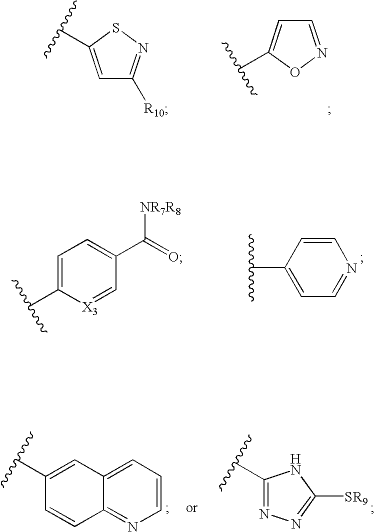
wherein:
X₃ is —CH— or —N—;
R₇ and R₈ are independently —H or an alkyl group or —NR₇R₈, taken together, is a nitrogen-containing non-aromatic heterocyclic group;
R₉ is an alkyl group; and
R₁₀ is —H or an alkyl group.

17. The method of claim 16 wherein Ring A is optionally substituted with one or more groups selected from —F, —Cl, —Br, —C1-C4 alkyl, C1-C4 alkoxy, —C1-C4 haloalkyl, C1-C4 haloalkoxy, —NH₂ or —CN.

18. The method of claim 17 wherein Ring A is unsubstituted; R₃ is a phenyl group or pyridyl group substituted with one or more substituents selected from —Br, —Cl, —F, —R^{e}, —OR^{e}, —CN, —COOR^{e}, —N(R^{e})₂, —CON(R^{e})₂, —NR^{e}COR^{f}, —NHCONH₂, —SO₂N(R^{e})₂; R₇ and R₈ are both —H and R₉ is methyl; and each R^{e} and R^{f} are independently —H, an alkyl group or a substituted alkyl group.

19. The method of claim 18 wherein R₃ is a phenyl ring substituted with one or more substituents selected from —Cl, —F, —R^{e}, —OR^{e}, —CN, —NH₂, —CONH₂ or —NHCOR^{f}.

20. The method of claim 19 wherein R₃ is a phenyl ring substituted with one or more substituents selected from —CH₃, —CH₂CH₃, —OCH₃, —CN, —F or —Cl.

21. The method of claim 19 wherein R₃ is a phenyl ring monosubstituted with —CH₃, —CH₂CH₃, —OCH₃, —CN, —F or —Cl and wherein the phenyl ring substituent is at the para position.

22. The method of claim 1 wherein the compound is represented by the following structural formula: 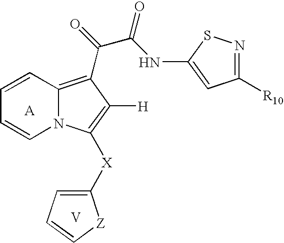
wherein:
Rings A and V are independently substituted or unsubstituted;
X is —CH₂—, —CH(CH₃)—, —O—, —NH— or —NCH₃—;
Z is —O—, —S—, —NR—, —C═C—, —CH═N—, —N═CH—, —N═N—; R is —H or C1-C4 alkyl; and R₁₀ is —H, an unsubstituted aliphatic group or a substituted aliphatic group.

23. The method of claim 22 wherein Ring A is unsubstituted or substituted with —F, —Cl, —Br, —C1-C4 alkyl, C1-C4 alkoxy, —C1-C4 haloalkyl, C1-C4 haloalkoxy, —NH₂ or —CN; Ring U is substituted with one or more groups selected from —CH₃, —CH₂CH₃, —F, —Cl, —CN, —OCH₃; and R₁₀ is —H, methyl or ethyl.

24. The method of claim 23 wherein Ring A is unsubstituted, Ring V is substituted with one or more groups R₁₁ and each R₁₁ is independently —CH₃, —CH₂CH₃, —OCH₃, —F, —Cl or —CN.

25. The method of claim 12 wherein R₂ is represented by the following structural formula:

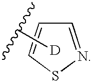

26. The method of claim 2 wherein the compound is:
2-(3-(4-cyanobenzyl)indolizin-1-yl)-N-(3-methylisothiazol-5-yl)-2-oxoacetamide;
2-(3-(4-chlorobenzyl)indolizin-1-yl)-N-(3-methylisothiazol-5-yl)-2-oxoacetamide;
2-(3-(4-chlorobenzyl)indolizin-1-yl)-2-oxo-N-(quinolin-6-yl)acetamide;
2-(3-(4-cyanobenzyl)-6-fluoroindolizin-1-yl)-N-(3-methylisothiazol-5-yl)-2-oxoacetamide;
2-(3-(4-cyanobenzyl)indolizin-1-yl)-2-oxo-N-(quinolin-6-yl)acetamide;
2-(3-benzylindolizin-1-yl)-N-(3-methylisothiazol-5-yl)-2-oxoacetamide;
2-(3-(4-fluorobenzyl)indolizin-1-yl)-N-(3-methylisothiazol-5-yl)-2-oxoacetamide;
2-(3-(4-methoxybenzyl)indolizin-1-yl)-N-(3-methylisothiazol-5-yl)-2-oxoacetamide;
2-(3-(4-cyanobenzyl)indolizin-1-yl)-N-(3-methylisothiazol-5-yl)-2-oxoacetamide;
2-(3-(4-chlorobenzyl)-8-methylindolizin-1-yl)-N-(3-methylisothiazol-5-yl)-2-oxoacetamide;
2-(3-(4-chlorobenzyl)-7-methylindolizin-1-yl)-N-(3-methylisothiazol-5-yl)-2-oxoacetamide;
2-(1-(4-chlorobenzyl)pyrrolo[1,2-a]quinolin-3-yl)-N-(3-methylisothiazol-5-yl)-2-oxoacetamide;
2-(3-(4-chlorobenzyl)indolizin-1-yl)-N-(2-carboxamidopyridin-5-yl)-2-oxoacetamide;
2-(3-(4-cyanobenzyl)indolizin-1-yl)-N-(1-ethyl-1H-pyrazol-5-yl)-2-oxoacetamide;
2-(3-(4-cyanobenzyl)indolizin-1-yl)-N-(4-phenylamide)-2-oxoacetamide;
2-(3-(4-cyanobenzyl)indolizin-1-yl)-2-oxo-N-(pyridin-4-yl)acetamide;
2-(3-(4-cyanobenzyl)-8-fluoroindolizin-1-yl)-N-(3-methylisothiazol-5-yl)-2-oxoacetamide;
2-(3-(4-cyano)-phenyl)carbonyl)indolizin-1-yl)-N-(3-methylisothiazol-5-yl)-2-oxoacetamide;
2-(3-(((4-cyano)-phenylmethyl)indolizin-1-yl)-1-(4-ethoxycarbonylpiperazin-1-yl)ethane-1,2-dione;
2-(3-(4-cyanophenoxy)indolizin-1-yl)-N-(3-methylisothiazol-5-yl)-2-oxoacetamide;
2-(3-(((4-cyano)-phenyl)methylamino)indolizin-1-yl)-N-(3-methylisothiazol-5-yl)-2-oxoacetamide;
2-(3-(1-(4-cyanophenyl)ethyl)indolizin-1-yl)-N-(3-methylisothiazol-5-yl)-2-oxoacetamide;
2-(3-(4-cyanobenzyl)-6-hydroxyindolizin-1-yl)-N-(3-methylisothiazol-5-yl)-2-oxoacetamide;
2-(3-(4-cyanobenzyl)-6-methylindolizin-1-yl)-N-(3-methylisothiazol-5-yl)-2-oxoacetamide;
2-(3-(4-cyanobenzyl)-6-nitroindolizin-1-yl)-N-(3-methylisothiazol-5-yl)-2-oxoacetamide;
2-(3-(3-cyanobenzyl)indolizin-1-yl)-N-(3-methylisothiazol-5-yl)-2-oxoacetamide;
2-(3-((5-chlorothiophen-2-yl)methyl)indolizin-1-yl)-N-(3-methylisothiazol-5-yl)-2-oxoacetamide; or
2-(3-((5-cyanothiophen-2-yl)methyl)indolizin-1-yl)-N-(3-methylisothiazol-5-yl)-2-oxoacetamide.

27. A method of treating a human subject with primary lung cancer or ovarian cancer comprising administering to the subject a compound represented by the following structural formula: 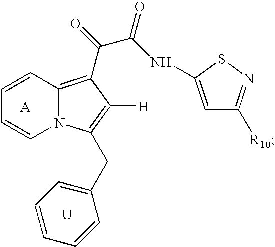
or a pharmaceutically acceptable salts thereof, wherein Rings A and U are independently substituted or unsubstituted and R₁₀ is —H, an unsubstituted aliphatic group or a substituted aliphatic group.

28. The method of claim 27 wherein the subject has epithelial ovarian carcinoma, ovarian germ cell carcinoma, or ovarian stromal carcinoma.

29. The method of claim 27 wherein the subject has squamous cell carcinoma, small cell carcinoma, adenocarcinoma, or large cell carcinoma.

30. The method of claim 27 wherein Ring A is unsubstituted or substituted with one or more substituents selected from —F, —Cl, —Br, —C1-C4 alkyl, C1-C4 alkoxy, —C1-C4 haloalkyl, C1-C4 haloalkoxy, —NH₂ or —CN; Ring U is substituted with one or more groups selected from —CH₃, —CH₂CH₃, —F, —Cl, —CN, —OCH₃; and R₁₀ is —H, methyl or ethyl.

31. The method of claim 30 wherein Ring U is monosubstituted with —CH₃, —CH₂CH₃, —F, —Cl, —CN, —OCH₃ and the Ring U susbstituent is at the para position.

32. A method of treating a human subject with primary lung cancer or ovarian cancer comprising administering to the subject a compound represented by the following structural formula: 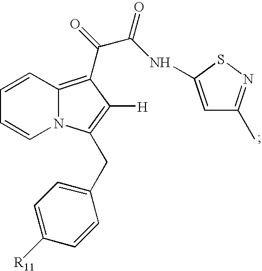
or a pharmaceutically acceptable salt thereof, wherein R₁₁ is —CH₃, —CH₂CH₃, —OCH₃, —F, —Cl or —CN.

33. The method of claim 32 wherein the subject is treated for epithelial ovarian carcinoma, ovarian germ cell carcinoma, or ovarian stromal carcinoma.

34. The method of claim 32 wherein the subject is treated for squamous cell carcinoma, small cell carcinoma, adenocarcinoma, or large cell carcinoma.
